(19) United States
(12) Patent Application Publication
(10) Pub. No.: US 2025/0340909 A1
(43) Pub. Date: Nov. 6, 2025

(54) METHOD OF METHANE CONVERSION USING AEROBIC METHANOTROPHS

(71) Applicant: Sogang Univesity Research & Business Development Foundation, Seoul (KR)

(72) Inventors: Jin Won LEE, Seoul (KR); Won Ho Jung, Seoul (KR); Suk Hyeong Cho, Gyeonggi-do (KR); Jeong Geol Na, Seoul (KR); Ye Jin Chol, Gyeonggi-do (KR)

(21) Appl. No.: 19/032,635

(22) Filed: Jan. 21, 2025

(30) Foreign Application Priority Data
May 2, 2024 (KR) 10-2024-0058464

(51) Int. Cl. C12P 5/02; B01D 53/84

(52) U.S. Cl. CPC C12P 5/023; B01D 53/84; B01D 2257/504; B01D 2258/05

(57) ABSTRACT

Disclosed herein is a methane conversion process that includes periodically injecting pure O₂ instead of air in the cultivation of aerobic methanotrophs. According to the process, methane and oxygen initially introduced can be recycled until they are completely removed, resulting in a final gas product consisting only of carbon dioxide and water. Carbon dioxide can be separated from the final gas product by simple cooling without the need for additional carbon dioxide capture processes such as CCU.

### CROSS-REFERENCE TO RELATED APPLICATIONS

This application claims the benefit and priority to Korean Patent Application No. 10-2024-0058464, filed on May 2, 2024. The entire disclosure of the application identified in this paragraph is incorporated herein by references.

### SEQUENCE LISTING

This application contains references to amino acid sequences and/or nucleic acid sequences which have been submitted concurrently herewith as the sequence listing XML file entitled “000387 us_SequenceListing.XML”, file size 14,901 bytes, created on 7 Jan. 2025. The aforementioned sequence listing is hereby incorporated by reference in its entirety pursuant to 37 C.F.R. § 1.52(e)(5).

### FIELD

The present disclosure was carried out under the support of the Ministry of Science and ICT, with the unique project number 1055001131, sub-project number 2015M3D3A1A01064929. The research management agency for this project is the National Research Foundation of Korea, and the project is titled “development of climate change mitigation technologies” with the research task named “Infrastructure research of C1 gas conversion technologies” The leading institution is Sogang University, and the research period spans from Jan. 1, 2023, to Dec. 31, 2023.

The present disclosure relates to technology for Methane Capture, Utilization, and Sequestration (MCUS) using aerobic methanotrophs. More specifically, the present disclosure pertains to a cultivation method that periodically supplies pure O₂ to easily separate carbon dioxide generated during methane oxidation without an additional CO₂ capture process and simultaneously obtain high-value products generated from aerobic methanotrophs, as well as a process applying the method.

### BACKGROUND

If the Earth's temperature rises by more than 2° C., natural disasters such as heatwaves, cold waves, heavy snowfall, typhoons, and wildfires occur. Therefore, with the goal of limiting the Earth's temperature rise to less than 1.5° C., major countries around the world have begun to participate in “carbon neutrality,” aiming to reduce greenhouse gas emissions, which cause abnormal weather, to “zero.” The EU, Germany, the UK, Japan, and others have declared carbon neutrality by 2050-2060, and South Korea is also taking active steps to achieve carbon neutrality by establishing a 2050 carbon neutrality scenario.

To achieve this goal, the development of various CO₂ reduction technologies, such as process electrification, CO₂ Capture and Utilization (CCU), and renewable energy generation, is actively progressing. Recently, as industry interest in methane emissions, in addition to carbon dioxide, has increased, the “Global Methane Pledge” was declared. Methane is one of the six major greenhouse gases defined by the Kyoto Protocol and has a global warming potential 21 times higher than carbon dioxide. Although there are technologies to convert methane into liquid fuel through chemical methods to reduce methane emissions, these methods have drawbacks. They are less economically and environmentally efficient than existing methane utilization processes due to extreme reaction conditions such as high temperatures and high pressures and the thermodynamic limitations caused by gas-phase equilibrium. Therefore, the biological conversion of methane using methanotrophs is relatively simple and can operate at room temperature and pressure. It can be proposed as an alternative that combines the reduction of methane, which would otherwise be burned and released into the atmosphere, with the production of value-added products.

Methane conversion technology using methanotrophs has been studied for various products such as organic acids, alcohols, and secondary metabolites, and shares a commonality with existing oxidative methane conversion in terms of the conversion under aerobic conditions, which requires oxygen. Aerobic methanotrophs can be divided into three main groups based on various assimilation pathways: Group I (ribulose monophosphate pathway (RuMP); Type I, Type X), Group II (serine pathway; Type II, Type III), and group III (Calvin-Benson-Bassham pathway; Type IV). Most studies on biological methane conversion have focused on methanotrophs such as Type I and Type II, which primarily produce high-value chemicals such as polyhydroxyalkanoates, single-cell proteins, methanol, organic acids, and ectoine. Despite numerous cultivation studies using methanotrophs, efforts to scale this technology to commercial-scale processes have been limited by low gas-liquid mass transfer, low process productivity, and conversion rates. Therefore, strategies to maximize methane conversion rates and recover as many products as possible are necessary.

According to the stoichiometry of the reaction involving methanotrophs (aCH₄+bO₂→cCell+dProducts+eCO₂+fH₂O), it can be seen that carbon dioxide is inevitably generated during cell growth and chemical production. This contradicts the claim that the biobased methane conversion reaction is environmentally friendly. Therefore, to achieve the inherent goal of biological methane conversion by methanotrophs, which is to convert methane into environmentally friendly high-value products, it is crucial not only to increase methane conversion rates and product yields but also to eliminate carbon dioxide emissions generated during the reaction.

In existing methane fermentation processes using aerobic methanotrophs, methane and air are used for methane oxidation. When gases discharged from the reactor are recirculated to maximize methane conversion rates, the accumulation of nitrogen lowers the partial pressure of carbon dioxide and methane in the final gas products, making the use of CCU essential.

### SUMMARY

With the problems in mind, the present inventors have succeeded in using aerobic methanotrophs to produce high-value chemicals from methane while capturing the emitted carbon dioxide without the need for additional technologies such as CCU, leading to the present disclosure.

Thus, the present disclosure aims to provide a methane conversion method including the following steps: a cultivation step of periodically supplying oxygen to a reaction unit containing methane and oxygen to culture aerobic methanotrophs; and a final gas separation step of separating a final gas containing carbon dioxide from the products generated by the aerobic methanotrophs.

The present disclosure relates to technology for Methane Capture, Utilization, and Sequestration (MCUS) using aerobic methanotrophs. More specifically, the present disclosure pertains to a cultivation method that periodically supplies pure O₂ to easily separate carbon dioxide generated during methane oxidation without an additional CO₂ capture process and simultaneously obtain high-value products generated from aerobic methanotrophs, as well as a process applying the method.

Below, a detailed description will be given of the present disclosure.

One aspect of the present disclosure is directed to a methane conversion method comprising the following steps: a cultivation step of periodically supplying oxygen to a reaction unit containing methane and oxygen to culture aerobic methanotrophs; and a final gas separation step of separating a final gas containing carbon dioxide from the products generated by the aerobic methanotrophs.

As used herein, the term “methanotroph” refers to a prokaryotic species that uses methane as a carbon source and chemical energy source for their life activities. Energy can be generated during the process of methane oxidation by methanotrophs, and methanol (CH₃OH) may be produced as a result of methane oxidation. Methanotrophs can be obligate aerobic, microaerobic, facultative anaerobic, or obligate anaerobic bacteria. For example, obligate aerobic or microaerobic methanotrophs can be used in the ectoine production process, but with no limitations thereto.

The methanotroph may be at least one selected from the group consisting of Methylomonas spp., Methylomicrobium spp., Methylobacter spp., Methylococcus spp., Methylosphaera spp., Methylocaldum spp., Methyloglobus spp., Methylosarcina spp., Methyloprofundus spp., Methylothermus spp., Methylohalobius spp., Methylogaea spp., Methylomarinum spp., Methylovulum spp., Methylomarinovum spp., Methylorubrum spp., Methyloparacoccus spp., Methylosinus spp., Methylocystis spp., Methylocella spp., Methylocapsa spp., Methylofurula spp., Methylacidiphilum spp., or Methylacidimicrobium spp., with no limitations thereto.

In an embodiment of the present disclosure, the methanotroph may belong to the genus Methylomicrobium.

In an embodiment of the present disclosure, the methanotroph may be the strain Methylomicrobium alcaliphilum 20Z.

The strain Methylomicrobium alcaliphilum 20Z is registered with the strain number DSM 19304 at the German Collection of Microorganisms and Cell Cultures (DSMZ) and can be obtained by purchase or distribution from DSMZ.

In an embodiment of the present disclosure, the methanotroph may be a strain obtained by removing the endogenous plasmid and deleting all or part of the ectoine hydroxylase gene (ectD) or the ectoine biosynthesis regulatory gene (ectR) from Methylomicrobium alcaliphilum 20Z.

The term “endogenous plasmid,” as used herein, refers to a plasmid that a cell naturally possesses before any external manipulation is performed. Endogenous plasmids can be linear or circular and may include genes that positively or negatively affect the life activities of the cells containing the endogenous plasmid. For example, methanotrophs may possess doeA, ectD, or ectR genes on endogenous plasmids, in addition to those present in their genome.

The term “ectoine hydrolase gene” (doeA), as used herein, refers to a gene encoding ectoine hydrolase, which is used by halophilic or halotolerant microorganisms to degrade ectoine for a carbon and nitrogen source. The ectoine hydrolase expressed from doeA hydrolyzes ectoine to convert it into an amino ester, which then becomes the substrate for subsequent enzymes and ultimately gets broken down into amino acids. The gene doeA can also be referred to as eutD.

When doeA is deleted in methanotrophs, the methanotrophs cannot hydrolyze already produced ectoine, thus providing a foundation for increasing ectoine production through methanotrophs.

The term “ectoine hydroxylase gene (ectD),” as used herein, refers to a gene encoding ectoine hydroxylase, which adds a hydroxyl group (—OH) to ectoine with the consequent conversion into hydroxyectoine. The hydroxyectoine produced from ectoine by ectoine hydroxylase expressed from ectD can act as an osmoprotectant for the cell, similar to ectoine.

The term “ectoine biosynthesis regulatory gene” (ectR), as used herein, refers to a gene encoding a transcriptional repressor for the ectABC operon or the ectABC-ask operon, which encodes a series of enzymes that induce ectoine biosynthesis.

In an embodiment of the present disclosure, a suicide vector may be used to delete all or part of the genes doeA, ectD, and ectR included in the genome of methanotrophs.

The term “suicide vector,” as used herein, refers to a non-replicable vector carrying a suicide gene that induces the deletion of a target gene from the host cell chromosome through homologous recombination and counterselection.

Specifically, sequences of the upstream and downstream regions of the gene to be deleted are inserted into the suicide vector and introduced into the host cell to induce homologous recombination with the host cell's chromosome. After transformation, culturing the host cells in a medium containing a substance that induces counterselection allows for the selection of transformed cells in which the suicide vector homologously recombined into the chromosome has been excised from the chromosome through subsequent homologous recombination. consequently, the genome of the final selected host cells will have all or part of the target gene deleted, effectively eliminating the function of the target gene through the suicide vector.

For example, by recombining the sequences of the upstream and downstream regions of the doeA gene into a suicide vector, and then performing homologous recombination and counterselection with the host cell chromosome, the doeA gene can be deleted from the host cell's genome.

The term “suicide vector” can be used interchangeably with “suicide plasmid.”

In an embodiment of the present disclosure, the methanotroph may be the strain Methylomicrobium alcaliphilum 20ZDP3.

In an embodiment of the present disclosure, a strain of Methylomicrobium alcaliphilum 20Z strain from which the endogenous plasmid has been removed and in which all or part of ectD and ectR have been deleted, without deletion of all or part of doeA, may be referred to as the Methylomicrobium alcaliphilum 20ZDP2 strain. A strain from which all or part of doeA has additionally been deleted, along with the removal of the endogenous plasmid and all or part of ectD and ectR, may be referred to as the Methylomicrobium alcaliphilum 20ZDP3 strain.

The Methylomicrobium alcaliphilum 20ZDP2 strain and the Methylomicrobium alcaliphilum 20ZDP3 strain have been deposited with the Korea Research Institute of Bioscience and Biotechnology (KRIBB) under accession numbers KCTC19047P (International accession number: KCTC16115BP) and KCTC19076P (International accession number: KCTC16116BP), respectively.

The reaction unit may have a volume of 250,000 to 400,000 L, for example, 311, 119.4 L, but is not limited thereto.

The methane may be included at 50 to 100% by volume of the reaction unit, preferably at 50 to 90%, 60 to 100%, 60 to 90%, 70 to 100%, or 70 to 90% by volume, for example, at 90% by volume, but is not limited thereto.

In the cultivation step, oxygen may be supplied such that the partial pressure of oxygen in the reaction unit is 1 to 20% of the partial pressure of methane in the reaction unit, preferably 5 to 20%, 10 to 20%, or 15 to 20%, for example, 20%, but with no limitations thereto.

The supply cycle of oxygen may be determined based on the degree of oxygen consumption in the reactor, for example, when all the oxygen in the reactor is consumed. The supply cycle may be 7 to 12 hours depending on the process conditions, but is not limited thereto.

The reaction unit may comprise 2 to 10 reactors, but with no limitations thereto. By operating multiple batch reactors with a time lag, a continuous methane conversion process can be realized from the perspective of the entire reaction unit.

Therefore, the number of reactors can be adjusted according to the methane assimilation rate of the methanotrophs used in the process. When using methanotrophs with a fast methane assimilation rate, a continuous methane conversion process can be realized with a smaller number of reactors due to the faster methane conversion rate per reactor unit.

The oxygen supplied to the reaction unit may be pure O₂, without other components. Supplying pure oxygen instead of air to the reaction unit can prevent the accumulation of nitrogen in the reaction unit.

The cultivation step may further include a nitrogen source supply step that supplies a nitrogen source to the reaction unit, but with no limitations thereto.

The term “nitrogen source,” as used herein, refers to a substance that can provide nitrogen compounds to the cells. By adding a nitrogen source to the medium in which the cells are cultured, the cells can utilize the nitrogen compounds in the medium, thereby achieving higher growth rates of the methanotroph strain and increased production of the product.

The nitrogen source supply step may continuously supply nitrogen at a concentration of 1 to 10 g/L per 1 g/L of aerobic methanotroph, preferably at concentrations of 1 to 9 g/L, 1 to 8 g/L, 1 to 7 g/L, and 1 to 6 g/L, for example, at a concentration of 1 to 5 g/L, but with no limitations thereto.

The nitrogen source may be selected from one or more of the group consisting of potassium nitrate (KNO₃), sodium nitrate (NaNO₃), ammonium chloride (NH₄Cl), ammonium sulfate ((NH₄)₂SO₄), yeast extract, urea, peptone, tryptone, or beef extract, but is not limited thereto.

The cultivation step may further include a step of continuously supplying an alkaline solution to adjust the pH of the reaction unit, but with no limitations thereto.

The alkaline solution may be at least one selected from the group consisting of NaOH, NH₄OH, or KOH, but is not limited thereto.

The product may include at least one from the group consisting of ectoine, methanol, PHA (polyhydroxyalkanoate), PHB (polyhydroxyvalerate), PHBV (poly(3-hydroxybutyrate-co-3-hydroxyvalerate)), microbial protein, biodiesel precursor, lactic acid, butyric acid, acetic acid, muconic acid, succinic acid, 3-hydroxypropionic acid, 2,3-butanediol, putrescine, cadaverine, and sesquiterpene, but is not limited thereto.

The term “capture,” as used herein, refers to the selective separation of a specific gas from a gaseous mixture. By culturing methanotrophs that use methane as the main energy source, methane in the reactor can be captured and converted into high-value products such as ectoine.

The stoichiometric equations for cell growth and ectoine production by methanotrophs can be represented as shown in Equations 1 to 3 below. Nitrogen is essential for all life activities of methanotrophs (Equation 1), and extra nitrogen is particularly required for ectoine biosynthesis (Equation 2). Thus, the addition of a nitrogen source can promote the ectoine biosynthesis by methanotrophs. This reaction can also be interpreted by adding the reaction of methane and oxygen, i.e., mineralization, which generates carbon dioxide (Equation 3).

$${CH}_{4} + \frac{3}{8} {O}_{2} + \frac{1}{4} {NO}_{3}^{-} \to \frac{1}{4} {C}_{4} {H}_{8} {O}_{2} N + {H}_{2} O \qquad [ Equation 1 ]$$

$${CH}_{4} + \frac{1}{4} {O}_{2} + \to \frac{1}{3} {NO}_{3}^{-} \to \frac{1}{6} {C}_{6} {H}_{10} {N}_{2} {O}_{2} + \frac{7}{6} {H}_{2} O \qquad [ Equation 2 ]$$

$${CH}_{4} + 2 {O}_{2} \to 2 {H}_{2} O + {CO}_{2} \qquad [ Equation 3 ]$$

The methane conversion method may further include a recycling step of re-supplying the medium separated from the product back into the reaction unit, but with no limitations thereto.

The cultivation step may be carried out until all the methane contained in the reaction unit is consumed, but with no limitations thereto.

The final gas may consist only of water and carbon dioxide, but is not limited thereto.

The present disclosure relates to a methane conversion process that includes periodically injecting pure O₂ instead of air in the cultivation of aerobic methanotrophs. According to the present disclosure, methane and oxygen initially introduced can be recycled until they are completely removed, resulting in a final gas product consisting only of carbon dioxide and water. Carbon dioxide can be separated from the final gas product by simple cooling without the need for additional carbon dioxide capture processes such as CCU.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a genetic map of a suicide vector constructed to delete doeA from the genome of a methanotroph strain according to an embodiment of the present disclosure;

FIG. 2 is a photographic image for whether or not doeA was deleted in a strain after homologous recombination of the upstream region of doeA in the suicide vector with doeA of the genome of the methanotroph strain and counterselection against sucrose according to an embodiment of the present disclosure, as analyzed by agarose gel electrophoresis;

FIG. 3 is a photographic image for whether or not doeA in a strain was deleted in a strain after homologous recombination of the downstream region of doeA in the suicide vector with doeA of the genome of the methanotroph strain and counterselection against sucrose, according to an embodiment of the present disclosure, as analyzed by agarose gel electrophoresis;

FIG. 4A is a plot of cell growth compared according to the amount of nitrogen source supplied in an embodiment of the present disclosure;

FIG. 4B is a plot of ectoine production compared according to the amount of nitrogen source supplied in an embodiment of the present disclosure;

FIG. 5A is a schematic diagram of a conventional methane fermentation process in which methanotrophs are cultured by injecting air, according to an embodiment of the present disclosure;

FIG. 5B is a schematic diagram of an MCUS methane fermentation process in which methanotrophs are cultured by injecting pure O₂, according to an embodiment of the present disclosure;

FIG. 6A is a plot of methanotroph cell growth and gas distribution in the reactor as measured using the conventional gas replacement method with an initial gas composition of air:methane=7:3, according to an embodiment of the present disclosure;

FIG. 6B is a plot of methanotroph cell growth and gas distribution in the reactor as measured under periodical injection of pure oxygen, with an initial gas composition of air:methane=7:3, according to an embodiment of the present disclosure;

FIG. 6C is a plot of methanotroph cell growth and gas distribution in the reactor as measured under periodical injection of pure oxygen, with an initial gas composition of air:methane=3:7, according to an embodiment of the present disclosure;

FIG. 7A is a schematic diagram of an ectoine production process using MCUS, according to an embodiment of the present disclosure; and

FIG. 7B is a schematic diagram showing the optimized operating conditions in an ectoine production process using MCUS, according to an embodiment of the present disclosure.

### DETAILED DESCRIPTION

Hereinafter, the present disclosure will be described in more detail with reference to the following Examples, which are set forth to merely illustrate the present disclosure, and but not to be construed to limit the scope of the present disclosure.

Throughout the specification, unless otherwise specified, “%” used to indicate the concentration of a specific substance means (weight/weight) % for solid/solid, (weight/volume) % for solid/liquid, (volume/volume) % for liquid/liquid, and (volume/volume) % for gas/gas.

### Example 1. Construction of Methylomicrobium alcaliphilum 20ZDP3 Strain with Ectoine Hydrolase Gene doeA Deleted therefrom

The strain Methylomicrobium alcaliphilum 20Z, a type of aerobic methanotroph, produces ectoine as a mechanism to withstand osmotic pressure when exposed to a saline environment. The ectoine hydrolase expressed by the doeA gene hydrolyzes ectoine into N-α-acetyl-L-2,4-diaminobutyrate.

To inhibit the degradation of ectoine observed after a specific growth point, the doeA gene was deleted in the Methylomicrobium alcaliphilum 20ZDP2 strain, which is a Methylomicrobium alcaliphilum 20Z strain (DSM 19304, DSMZ, Germany) which lacks the genes ectD (mediating the conversion of ectoine to hydroxyectoine) and ectR (a transcriptional repressor of the ectoine operon involved in ectoine biosynthesis). The gene deletion was achieved through homologous recombination with a recombinant suicide vector constructed by recombining upstream and downstream fragments of doeA with the suicide vector pK19mobsacB.

The specific steps for constructing the Methylomicrobium alcaliphilum 20ZDP3 strain are as follows:

1-1. Construction of Suicide Vector with Recombined Upstream and Downstream Fragments of doeA

pK19mobsacB was digested with the restriction enzymes HindIII and EcoRI. The upstream and downstream fragments of doeA were obtained through polymerase chain reaction (PCR) and then ligated into pK19mobsacB using 5× infusion (Takara). The nucleotide sequences of the upstream and downstream fragments of doeA, as well as the primer sequences used for amplification, are shown in Table 1.

TABLE 1

| SEQ | | |
|---|---|---|
| ID | | |
| NO: | Name | Sequence listing (5′-> 3′) |
| 1 | doeA Upstream | atgaaagtctttgaacaatgggaatccgaaattcgcggctactgccgtgtg |
| | gene | tatcctaccgttttcaaatcggcctccaatgcccgtcaagtggatgagtcgg |
| | | gaaaatcctatatcgacttcttcgcaggcgccggtgtactcaatttcggcca |
| | | caacaacccgttgatgaaaaaagccttgatcgattttatcgaggcggatg |
| | | gcgtagcccacagtctggacacctacaccacggcgaaacgcgattttatc |
| | | gaagccttcgccaattccgtgcttaaaccgcgaaaaatgaattacaaaat |
| | | gcaattcatgggaccgaccggaactaatgcggtcgaaaccgcgctgaa |
| | | attggcgcgcaaggtcaccgggcgccgttcgattattgcgttcaatcacgg |
| | | ttttcacggcatgacactgggctcgctggcctgcacggcaaaccaatattt |
| | | ccgcaatgccgccggcgtacccctggaatatgttcgtcatgaacccttcgg |
| | | ctgcgaaaagccttgtatcggttgccaattaggctgtggcctggaaactatc |
| | | gatcagttgcgcgcgcaattttccgattcgtcgagcggcctggaaccgccg |
| | | gcggcgtttttagtcgaaccgattcaagccgaaggcggcgtcaatgtagc |
| | | cagtcgggaatggttgcacgccgtgcaaaaaatggcgcacgatctaggc |
| | | gcgctattgatttttgacgacatccaggcaggttgcgggcgcaccggtcaa |
| | | tatttcagttttgacggcatggatttggaaccggacattatcacattagccaa |
| | | aggcatcggcggcttcggcacgccgctggcgatgaatttggtcaagcctg |
| | | aacacgaccagcattggcagcccggcgaacataccggcactttcagag |
| | | gtcaaggcttctccttcgtcgcgggcaaagtggcgctgtcctatttcgacga |
| | | tgacgcattgatgaacgatgtcaagctgaaaggcactaagatgcagcag |
| | | cacctcgaagacttggggaacgcttcggtcaagggcggtttcaagtgcg |
| | | cggcaaaggcatgatgcaagggctggacatcgccgatggcgccttggc |
| | | gaaaaacatcgtcaatctttgcttcgaacgcggactactactcagcgcctg |
| | | cggaacgggggcaaggtcatcaaaatgattccgccgctaacgattccg |
| | | gaagccgatctgctcgaaggtttaacgattttctcgggcgtcttagcggatg |
| | | ctttggaggcctt |
| 2 | doeA | tcaagattatgaattaaccatgaagaacatgaagaattagattgttgacttt |
| | Downstream gene | gcaaggagggttactcaatggctaccacgctccagaaactgtgaaagtat |
| | | ataggctttttcttcatggtgaataaattttttatgatgtattcactaacttacgg |
| | | cctcaacagcgtgggagcgatgaataatgatattcctaatttagcttattca |
| | | acggagaacgaaactaatgagcatacaagttattaatccatccaacggc |
| | | gaatcattgggcataagcccggaactcgacgccgccgctcgcgatgctg |
| | | cgctcgatcgcgcgcaaatagcctatgccgattggagaaaccaatccttt |
| | | gcgcaacgcgcgcaaatgttgcgtaaaatcgctcagcgtttacgcgatga |
| | | tgtagaatcgctggcaccgctgatgaccttggagatgggcaaaccgatca |
| | | aggaggcccgtgctgaagtgatgaaggcagcgctctgcgccgaccatta |
| | | cgccgaacatggcgagagctatcttgccccggatacattggcatctgacg |
| | | ccagtcttagctatgtgcaatatctgcctttgggcgttgtactgggcattttgc |
| | | cttggaacgcaccattttggctggcttttcgcttttgcgccccggcattgatgg |
| | | ccggaaacacctgcctgatgaagcatgacccgcatgttccggcctgcgc |
| | | cgaagcaatcgcgcggctatttgagcagagcggagccccggccggtcta |
| | | ttgcaaaatctacccttgtcgaacaccgatgtagaagcggtcattcgcgat |
| | | ccacgcgtacaagccgtctcgttgaccggttcggatagagccggcactgt |
| | | cgtggcctcgatcgccggtgctgaaatcaaaccggtcgttctggaattggg |
| | | cggctccgatccctgcatcgtactggccgatgcagatctggacaaagccg |
| | | ccgatgtgattacgctgtcgcgcatcatcaatgccggccaatcttgtatcgc |
| | | cgccaaacgcatcattgtcgaagccggcattcatgatgaactcgtgacac |
| | | ggcttgaagaacgtctaagcaagttgaaactgggcgacccgaatgagga |
| | | aagcacagacatcggccccttggcgcgagaagacttgcggctaaacctg |
| | | catcgtcaggttcaagaaacgattgccgccggcgcacaatgccgactcg |
| | | gcggaaaaataccggacgggccgggctatttttatcccgtgaccttgctaa |
| | | ccggcgttacgaacgacatgacggccgcccgagaagaaaccttcggcc |
| | | cggtcgctgtagttattaaagccgaagatcaagatgaagcgatgcgcata |
| | | gccaacgacagtcaatatggtctagccgccagcatctggacggaacggt |
| | | cgcgaggcgaagcgctggctcggcaattggaaaccggccaagttgcgg |
| | | tcaacggcatcgtcaaaaccgaccccagactaccaagcggcggcgtca |
| | | agcgctcaggactcggccgcgaactggggccgcacggcatgcacgaat |
| | | ttgtcaacgcgcagcaggtttggctgggttaa |
| 3 | doeA Upstream | tgacatgattacgccaagcttatgaaagtctttgaacaatgggaa |
| | forward Primer | |
| 4 | doeA Upstream | ccctccttgcaaggcctccaaagcatccg |
| | Reverse Primer | |
| 5 | doeA | tggaggccttgcaaggagggttactcaatggc |
| | Downstream | |
| | forward Primer | |
| 6 | doeA | aaaacgacggccagtgaattcttaacccagccaaacctgctgc |
| | Downstream | |
| | Reverse Primer | |

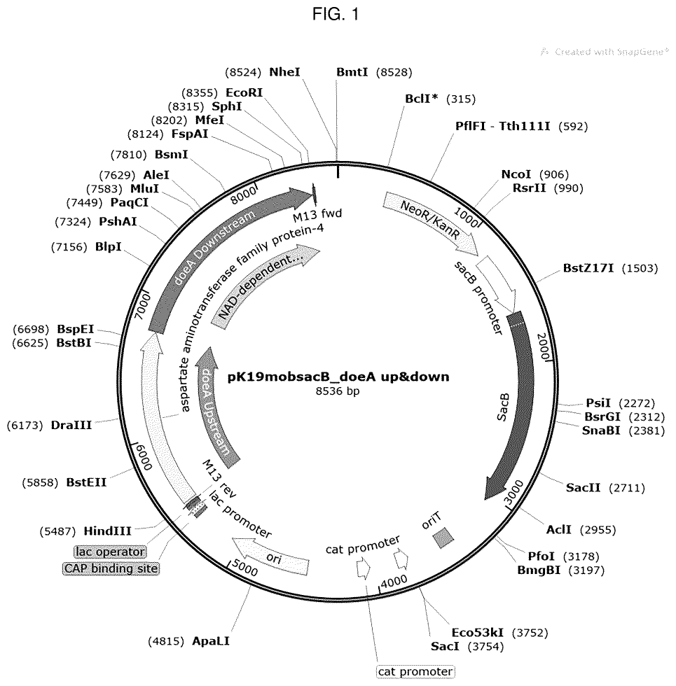

The genetic map of the recombinant suicide vector with the upstream and downstream fragments of doeA recombined into the suicide vector pK19mobsacB is shown in FIG. 1.

The constructed recombinant suicide vector was then introduced into E. coli DH10 competent cells by heat shock to confirm the stability of the recombinant suicide vector.

1-2. Homologous Recombination of Recombinant Suicide Vector into Chromosome of the Methanotroph Strain

The E. coli S17-1 λ pir strain was transformed by introducing the recombinant suicide vector into competent cells thereof by heat shock. The E. coli S17-1 λ pir strain transformed with the recombinant suicide vector and the Methylomicrobium alcaliphilum 20ZDP2 strain were each cultured in 50 mL until the optical density at 600 nm reached 0.5. The cells were then harvested by centrifugation at 4,000 rpm and 4° C. for 10 minutes.

The harvested cells of the two strains were resuspended and mixed in 200 μL of broth, and the mixture was applied to a nitrocellulose filter with a 0.2 μm pore size, which was placed on DSMZ plates (2 g/L NaCl, 15% nutrient broth, 1% (v/v) methanol). The cells were incubated statically at 30° C. for two days to induce homologous recombination of the gene carriers through junction between the two strains. The cells on the filter were collected using a loop and suspended in a tube containing 200 μL of DSMZ medium (1% (v/v) methanol).

To select only the Methylomicrobium alcaliphilum 20ZDP2 strain that had successfully integrated the recombinant suicide vector, the suspension was spread on DSMZ plates (1% (v/v) methanol) containing the antibiotic kanamycin (50 μg/mL) for indicating the insertion of pK19mobsacB, and nalidixic acid (10 μg/mL), which is toxic to E. coli. The plates were incubated statically at 30° C. for 5 days. To confirm the results of antibiotic selection, PCR was performed with two pairs of primers considering the homologous recombination of the upstream and downstream fragments of doeA. Cells with homologous recombination at the upstream or downstream fragments of doeA were identified.

The nucleotide sequences of the two pairs of primers used for PCR to confirm the results of antibiotic selection are shown in Table 2.

TABLE 2

| SEQ ID | | Sequence |
|---|---|---|
| NO: | Name | listing (5′-> 3′) |
| 7 | doeA Upstream Integration_check Forward | atgcttccggctcgtatgtt |
| | Primer | |
| 8 | doeA Upstream Integration_check Reverse | tccggatcgctgattacgac |
| | Primer | |
| 9 | doeA Downstream Integration_check | gcgtcgaaggcgataaaacc |
| | Forward Primer | |
| 10 | doeA Downstream Integration_check | taagcccactgcaagctacc |
| | Reverse Primer | |

1-3. Removal of doeA from Methanotroph Strain Through Sucrose Counterselection of Homologous Recombination with Recombinant Suicide Vector

Single colonies homologously recombined with the upstream and downstream fragments of doeA were selected and each inoculated into 3 mL of DSMZ broth (1% (v/v) methanol) and then cultured with shaking for 3 days. Subsequently, the culture was spread on DSMZ solid plates (1% (v/v) methanol) containing 5% (w/v) sucrose and incubated statically at 30° C. for 5 days to induce counterselection with sucrose, leading to the release of the recombinant suicide vector and the removal of doeA from Methylomicrobium alcaliphilum 20ZDP2.

To confirm the release of the recombinant suicide vector, antibiotic selection was conducted. Specifically, a 100-column pattern diagram was attached on each of the DSMZ plates (1% (v/v) methanol) with or without 5% (w/v) kanamycin, and 100 single colonies cultured on DSMZ plate (1% (v/v) methanol) containing 5% (w/v) sucrose were randomly selected and inoculated onto the DSMZ plates (1% (v/v) methanol) with or without kanamycin.

After 5 days of static incubation at 30° C., colonies that did not survive on the kanamycin-containing DSMZ (1% (v/v) methanol) plate but survived on the DSMZ plate (1% (v/v) methanol) without kanamycin were selected, indicating the removal of the recombinant suicide vector and doeA from Methylomicrobium alcaliphilum 20ZDP2.

To finally confirm the removal of doeA in the genome of the selected colonies, PCR was performed to observe the amplification of the doeA gene fragment. The sequences of the primers used for PCR to confirm the removal of doeA are shown in Table 3.

TABLE 3

| SEQ ID | | Sequence |
|---|---|---|
| NO: | Name | Listing (5′-> 3′) |
| 11 | doeA Elimination_check | gcttcgaacgcggactacta |
| | Forward Primer | |
| 12 | doeA Elimination_check | cgctgttgaggccgtaagtt |
| | Reverse Primer | |

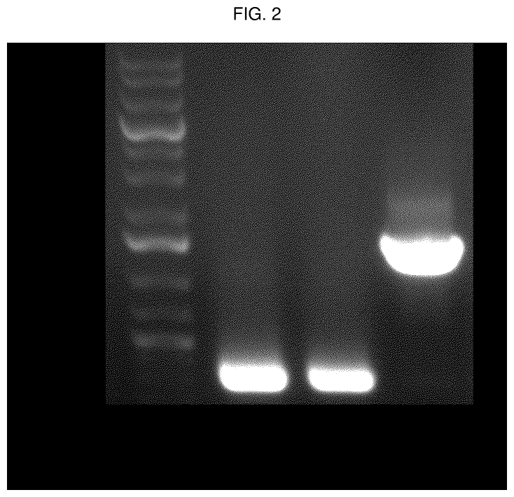

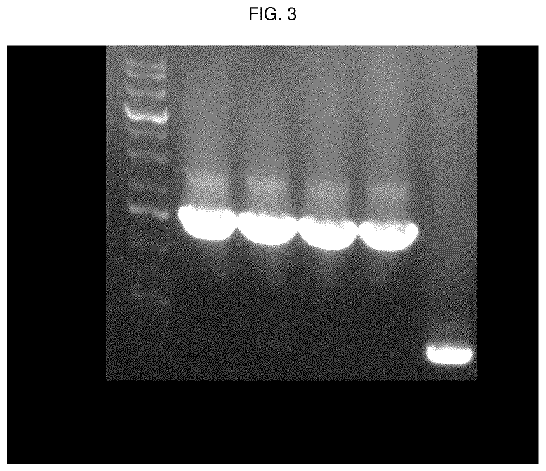

The gene fragments amplified by PCR were confirmed by agarose gel electrophoresis, and the results are shown in FIGS. 2 and 3. A radiation band appeared at 1490 bp when doeA was not removed and at 514 bp when doeA was removed.

The results confirmed that doeA was successfully deleted in both cases of homologous recombination with the upstream or downstream fragment of doeA in the Methylomicrobium alcaliphilum 20ZDP2 strain. Consequently, the Methylomicrobium alcaliphilum 20ZDP3 strain with the deletion of doeA was obtained from the Methylomicrobium alcaliphilum 20ZDP2 strain. The obtained Methylomicrobium alcaliphilum 20ZDP3 strain was stored in a deep freezer using 10% (v/v) DMSO as a cryoprotectant at an optical density of 1-2 at the wavelength of 600 nm.

### Example 2. Cultivation of Aerobic Methanotroph

The Methylomicrobium alcaliphilum 20ZDP3 strain was cultured in the Methylomicrobium medium shown in Table 4.

TABLE 4

| Methylomicrobium medium composition | (g/L) |
|---|---|
| NaCl | 30 |
| MgSO₄•7H₂O | 0.2 |
| KNO₃ | 1~5 |
| CaCl₂•2H₂O | 0.02 |
| Trace element (ml) | 1 |
| 1M NaHCO₃ (ml) | 50 |
| 1M Na₂CO₃ (ml) | 5 |
| Phosphate Buffer (ml) | 20 |

The phosphate buffer (PB) was prepared and diluted as shown in Table 5, and the trace element solution was prepared and diluted as shown in Table 6.

TABLE 5

| Phosphate buffer 50x composition | (g/L) |
|---|---|
| KH₂PO₄ | 14 |
| Na₂HPO₄*12H₂O | 30 |

TABLE 6

| Trace element 1000x composition | (mM) |
|---|---|
| EDTA | 17.11 |
| CuCl₂*5H₂O | 15 |
| FeSOV*7H₂O | 7.19 |
| ZnSO₄*7H₂O | 0.35 |
| H₃BO₃ | 0.49 |
| NiCl₂*6H₂O | 0.06 |
| COCl₂*6H₂O | 0.84 |
| Na₂MoO₄ | 0.15 |
| MnCl₂*4H₂O | 0.49 |
| Sodium tungstate | 0.05 |

The strain inactivated and stored in a deep freezer was revived by culturing in 50 mL of DSMZ broth for two days, followed by one passage. The strain was then inoculated into 50 mL of DSMZ broth in a 300 mL Erlenmeyer flask at an initial optical density of 0.2. The culture was incubated for 120 hours at 30° C. with shaking at 230 rpm.

### Example 3. Measurement of Cell Growth and Ectoine Production According to Nitrogen Source Supply

The strain pre-cultured under the same medium composition and conditions as in Example 2 was passaged in 300 mL of Methylomicrobium medium containing 30 g/L NaCl and 1% methanol (v/v). A dissolved oxygen probe and a pH probe were assembled into a 5 L bioreactor that was equipped with a stirrer on the bottom. The medium was added to the bioreactor and sterilized before the experiment was conducted. During the main cultivation, the stirring speed was controlled between 300 and 650 rpm at 30° C. The supply gas was adjusted to 30% methane (v/v) and 70% air at 0.1 to 0.52 vvm to control dissolved oxygen. The pH was adjusted to a range of 8.9 to 9.1 using 5N sulfuric acid and 5N sodium hydroxide solutions. The initial inoculation concentration was set to 0.04 g/L (optical density of 0.2).

To observe the changes in cell growth and ectoine production according to the nitrogen source supply, experiments were conducted using the bioreactor under the same conditions. KNO₃ was added at concentrations of 1 g/L, 2 g/L, and 5 g/L, respectively, each time the cell concentration increased by 2 g/L. Additionally, 1× trace elements were added along with KNO₃ before sampling.

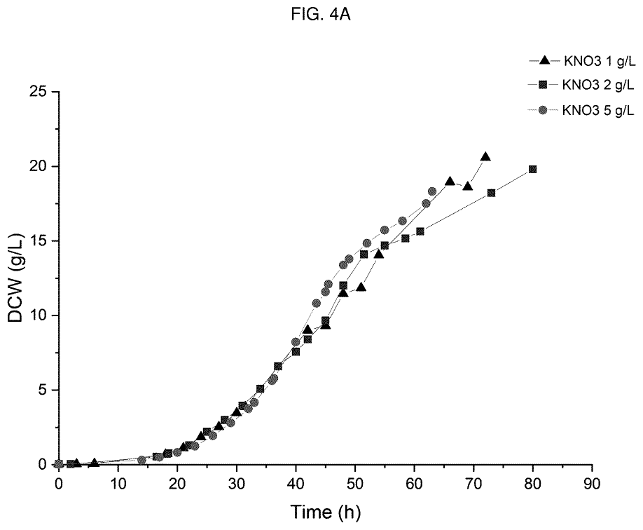

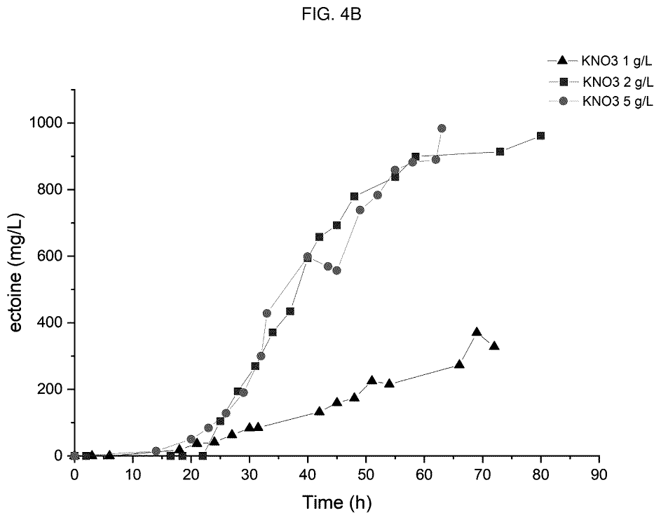

As shown in FIGS. 4A and 4B, cell growth was not significantly affected by the amount of nitrogen in the medium. However, ectoine production increased proportionally to cell production when KNO₃ was provided at 1 g/L or more per 1 g/L of cell production. It was also observed that up to 5 g/L of KNO₃ did not inhibit cell growth and ectoine production.

Based on this example, kinetic modeling and reactor modeling were applied to optimize cell growth and ectoine production. This mathematical framework aims to create an environment that can closely simulate actual experiments through gas transport modeling.

### Example 4. Measurement of Cell Growth and Gas Distribution Changes in Reactor with Pure O₂ Injection

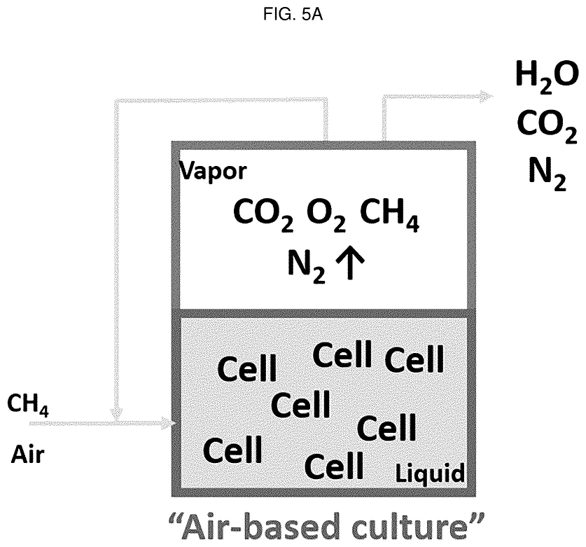

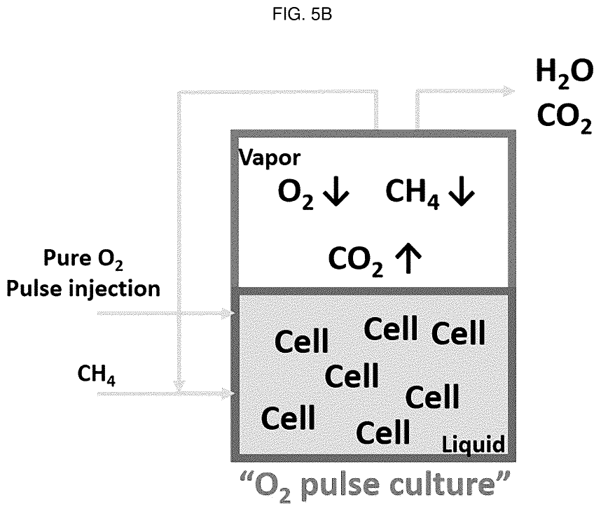

In conventional methane fermentation processes using methanotrophs, air is used as shown in FIG. 5A. However, in such cases, nitrogen accumulates in the reactor, requiring a separate CO₂ capture and utilization (CCU) technology to separate carbon dioxide from the mixed gas containing nitrogen at the end of the reaction. The economics of CCU depend on the concentration of carbon dioxide, and the concentration range of carbon dioxide in the exhaust gas of conventional methane fermentation processes is about 6-9%, which significantly increases the cost of CCU compared to typical post-combustion capture processes.

In the present disclosure, as shown in FIG. 5B, periodic injection of pure oxygen allows the separation of carbon dioxide produced in the metabolic processes of aerobic methanotrophs without a separate CCU process. The separation of pure carbon dioxide according to the embodiments of the present disclosure can be interpreted as the “production” of carbon dioxide that can be utilized in various industries, rather than as an “emission” that incurs processing costs. Thus, the Methane Capture, Utilization, and Sequestration (MCUS) technology based on biotransformation is defined.

To verify the feasibility of the MCUS process, cell growth and gas distribution changes were measured by culturing the pre-cultured strain under the same medium composition and conditions as in Example 2 in both the conventional methane conversion process and the MCUS process of the present disclosure. The gas supply for each process was carried out under the conditions shown in Table 7.

TABLE 7

| Operation | Gas composition | Detailed method |
|---|---|---|
| Conventional | Air:methane = 7:3 | Gas refresh every 12 hours |
| methane | (volume ratio) | |
| convention | | |
| process | | |
| MCUS (7:3) | Air:methane = 7:3 | Inject 25-50 ml of O₂ every |
| | (volume ratio) | 24 hours |
| MCUS (3:7) | Air:methane = 3:7 | Inject 50 ml of O₂ every 12 |
| | (volume ratio) | hours |

In the conventional methane conversion process, methanotrophs were cultured with an initial gas composition of 70% air and 30% methane. Every 12 hours, the gas was refreshed with a flow rate of 300 cc per minute for 4 minutes, maintaining the initial gas composition of 70% air and 30% methane to ensure consistency every 24 hours. In the MCUS (7:3) experimental group, the initial flask gas composition was set the same as the conventional methane conversion process (70% air and 30% methane), with pure oxygen injected at 25-50 ml intervals every 24 hours. Additionally, to observe the effects of varying the ratio of air to methane in the initial gas composition, in the MCUS (3:7) experimental group, the initial flask gas composition was set to 30% air and 70% methane, with pure oxygen injected at 50 ml intervals every 12 hours.

For all experiments, gas chromatography was used to analyze the gas composition in the headspace before gas replacement or pure oxygen injection. Liquid samples (1 ml) were taken to measure optical density using a spectrophotometer to compare cell growth under each condition. The gas analysis using gas chromatography was normalized with nitrogen to calculate the moles of each gas present in the headspace.

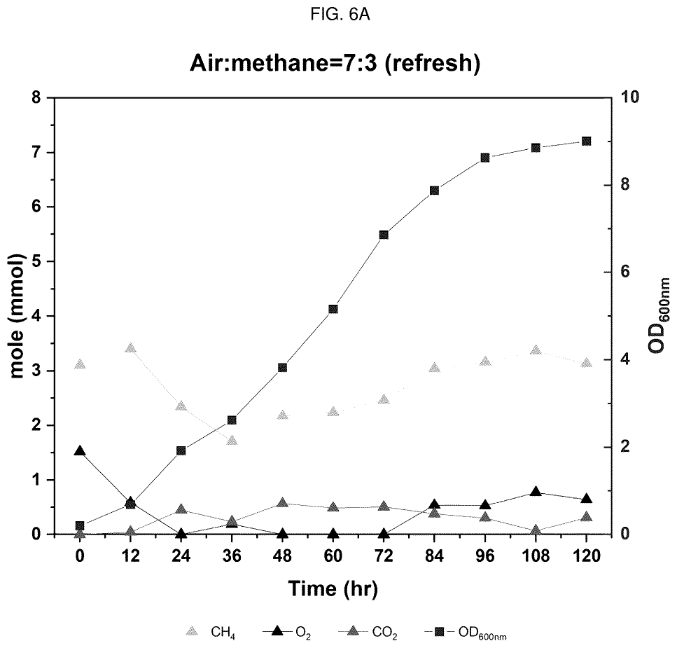

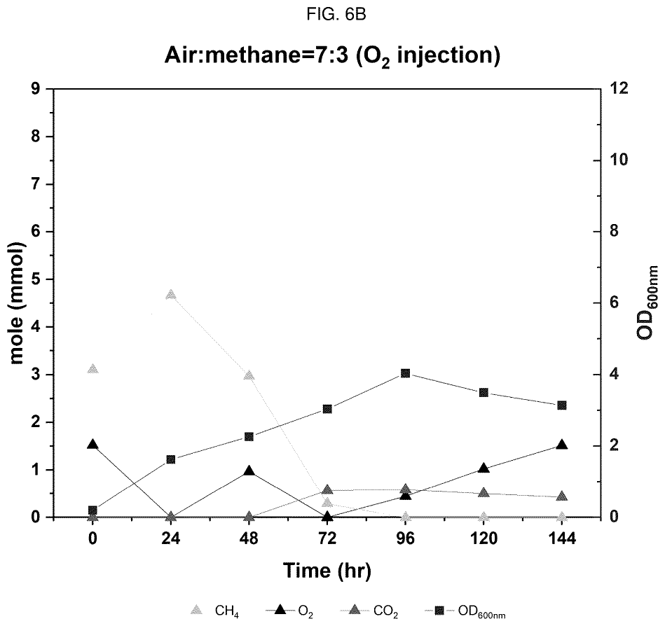

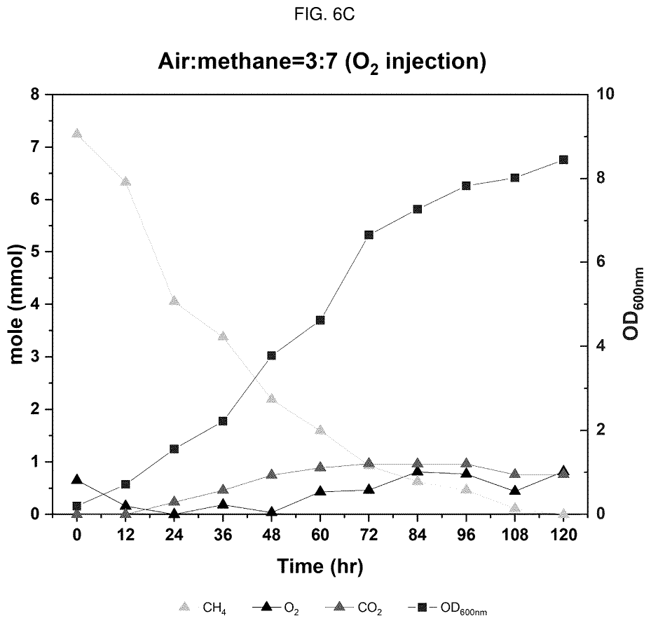

The changes in gas composition and optical density under each condition are shown in FIGS. 6A to 6C. The results indicated that, with the implementation of the MCUS process, cell growth continued until all methane was consumed. Particularly, in the MCUS process with a gas composition of 30% air and 70% methane, cell growth rates similar to those in the conventional methane conversion process were achieved. Ultimately, both methane and oxygen were completely consumed, leaving only carbon dioxide accumulated in the headspace. This confirmed the feasibility of the MCUS process, where periodic injection of pure oxygen into the reactor culturing aerobic methanotrophs results in a final gas composed only of water and carbon dioxide, with no nitrogen remaining.

### Example 5. Design of Methane Conversion Process Using Aerobic Methanotrophs

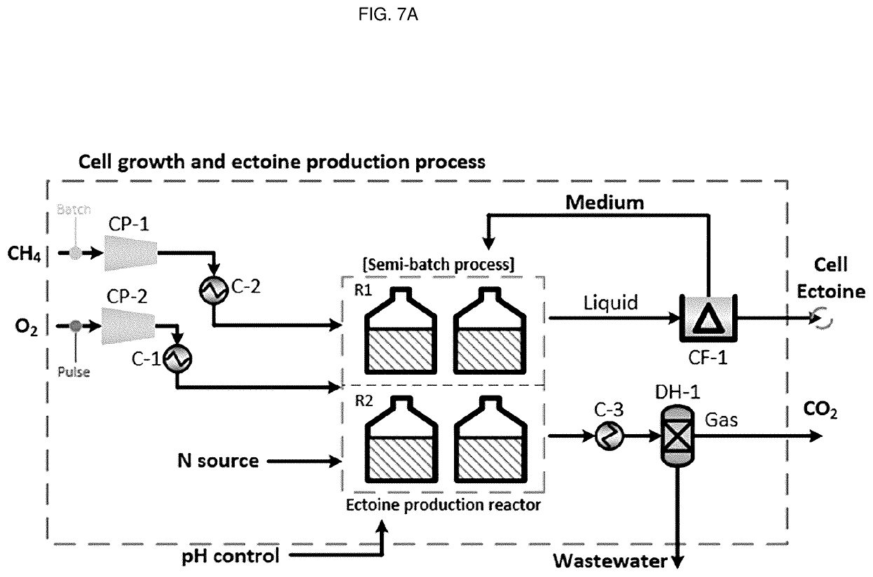

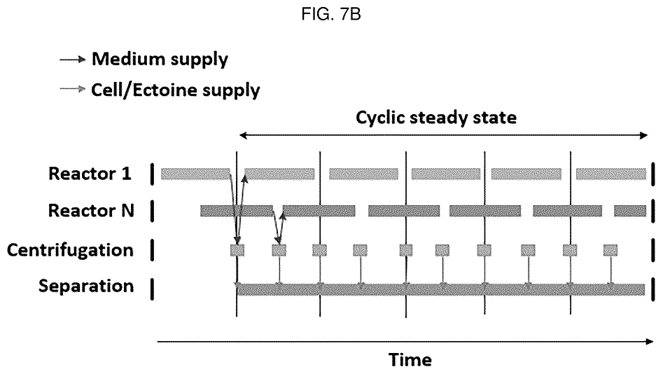

A basic schematic diagram of the reaction process for implementing MCUS is shown in FIG. 7a. The reaction unit consists of multiple reactors forming a module, with the supply of methane and oxygen regulated according to the operation of this module. The Methylomicrobium alcaliphilum 20ZDP3 strain was inoculated into each reactor at a concentration of 0.03 to 1.25 g/L and cultured under initial nitrogen-removed gas conditions, composed only of oxygen and methane (CH₄ 50-100%), at 30° C. and 1-5 bar. Additionally, an alkaline solution was continuously supplied to the reactor to adjust the nitrogen source and maintain the pH between 7 and 9 during the process. Methane and oxygen were supplied to the reactor through a compressor and cooler, with methane supplied in batches and oxygen supplied in pulses. Oxygen was supplied until the partial pressure of oxygen in the reactor reaches a maximum of 20%, and the time taken for the oxygen to be completely consumed in the reactor was measured to determine the periodic oxygen supply schedule.

After reaction for a sufficient time in the reactor, the gas cleared of both methane and oxygen was discharged from the rear end of the reactor while the water was saturated with pure carbon dioxide. By passing the discharged gas through a cooler and dehydration process, pure carbon dioxide could be obtained. The liquid products generated in the reactor, including cells, ectoine, water, and medium, could be separated into cell+ectoine/water+medium through centrifugation. The separated water and medium were then re-supplied to the reactor. The separated cells and ectoine were sent to a separate separation process where additional steps were taken to obtain ectoine separated from the cells.

FIG. 7b illustrates an example of optimized operation in the ectoine production process. Multiple batch reactors can be installed to run a continuous methane conversion process. The number of reactors in the reaction unit can range from 2 to 10, depending on the methane assimilation rate of the methanotrophs used in the process.

When multiple reactors are started with a certain time lag, the overall reaction unit can operate as a continuous methane conversion process rather than a batch process. Within a few hours after the initial reaction, a cyclic steady state is achieved.

### Example 6. Efficiency Comparison with Conventional Methane Conversion Processes

To verify the efficiency of the methane conversion process designed in Example 5, a comparison was made of the efficiency between a conventional methane conversion process that continuously supplies gas containing CH₄ (Process A) and the process of the present disclosure (Process B) under the conditions designed as shown in Table 8.

TABLE 8

| | Process A | Process B |
|---|---|---|
| | (Conventional | (Methane conversion |
| | methane conversion | process with periodic |
| | process) | oxygen supply) |
| Time taken to carry | 49 | 49 |
| out process (h) | | |
| Initial strain conc. | 1.25 | 1.25 |
| (g/L) | | |
| Initial gas | 70% air, 30% CH₄ | 10% O₂, 90% CH₄ |
| composition | | |
| Supplied gas | 70% air, 30% CH₄ | 100% O₂ |
| composition | | |
| Gas supply speed | 520.625 kg/h | Supplied to maintain O₂ |
| | (air 420.625 kg/h, | fraction of up to 20% of the |
| | CH₄ 100 kg/h) | CH₄ fraction in reactor |
| Reactor volume | 311119.4 L | 62223.4 L/reactor |
| No. of reactor | 1 | 5 |

For both Process A and Process B, a methane conversion reaction was designed to supply a total of 4900 kg of CH₄ over 49 hours in reactors of the same volume. In Process A, a mixed gas (70% air, 30% CH₄ by volume) was supplied at a rate of 420.625 kg/h to achieve a CH₄ supply rate of 100 kg/h. In Process B, an initial mixture of gas containing 4900 kg of CH₄ (10% O₂, 90% CH₄ by volume) was injected into the reactor, followed by periodic O₂ supply.

Specifically, Process B used five reactors, each ⅕ the size of the reactor in Process A. The reaction start times for each reactor were staggered by 2 hours to ensure continuous reactions, similar to Process A. The time for complete O₂ consumption was measured to periodically supply O₂, with O₂ supplied at intervals of approximately 7 hours to maintain an O₂ fraction of up to 20% of the CH₄ fraction in each reactor.

After 49 hours of methane conversion, the methane conversion rate, oxygen conversion rate, final cell concentration, and ectoine production were compared between the two processes, as shown in Table 9.

TABLE 9

| | Process A | Process B |
|---|---|---|
| | (Conventional | (Methane conversion |
| | methane conversion | process with periodic |
| | process) | oxygen supply) |
| CH₄ Conversion (%) | 40.06 | 100 |
| O₂ Conversion (%) | 93.97 | 100 |
| Final Cell Concentration | 6.26 | 13.27 |
| (g/L) | | |
| Ectoine Production (g/L) | 0.32 | 0.74 |

In Process A, continuous methane supply inside the reactor made it impossible to completely convert methane, resulting in a final methane conversion rate of 40.06%. In contrast, Process B could achieve 100% methane conversion in the reactor, as the reaction continued until all methane in the initial reaction was consumed. Furthermore, the final cell concentration in Process A was 6.26 g/L, whereas Process B achieved more than twice the final cell concentration at 13.27 g/L due to the higher methane conversion rate, resulting in over twice the efficiency in ectoine production during the same period.

### DEPOSIT INFORMATION

Depositary Authority: Korea Research Institute of Bioscience and Biotechnology (KCTC)
Accession Number: KCTC19047P
International Accession Number: KCTC16115BP
Deposit Date: Jan. 20, 2023
International Conversion Date: Oct. 29, 2024
Depositary Authority: Korea Research Institute of Bioscience and Biotechnology (KCTC)
Accession Number: KCTC19076P
International Accession Number: KCTC16116BP
Deposit Date: Mar. 8, 2023
International Conversion Date: Oct. 29, 2024

What is claimed is:

1. A method for methane conversion, comprising:
a cultivation step of periodically supplying oxygen to a reaction unit containing methane and oxygen to culture an aerobic methanotroph; and
a final gas separation step of separating a final gas containing carbon dioxide from a product generated by the aerobic methanotroph.

2. The method of claim 1, wherein the aerobic methanotroph is at least one selected from the group consisting of Methylomonas spp., Methylomicrobium spp., Methylobacter spp., Methylococcus spp., Methylosphaera spp., Methylocaldum spp., Methyloglobus spp., Methylosarcina spp., Methyloprofundus spp., Methylothermus spp., Methylohalobius spp., Methylogaea spp., Methylomarinum spp., Methylovulum spp., Methylomarinovum spp., Methylorubrum spp., Methyloparacoccus spp., Methylosinus spp., Methylocystis spp., Methylocella spp., Methylocapsa spp., Methylofurula spp., Methylacidiphilum spp., or Methylacidimicrobium spp.

3. The method of claim 1, wherein the methane is included at 50 to 100% by volume of the reaction unit.

4. The method of claim 1, wherein the cultivation step comprises supplying oxygen such that the partial pressure of oxygen in the reaction unit is 1 to 20% of the partial pressure of methane in the reaction unit.

5. The method of claim 4, wherein the cultivation step comprises supplying oxygen when all the oxygen in the reaction unit is consumed.

6. The method of claim 1, wherein the cultivation step further comprises a nitrogen source supply step of supplying a nitrogen source to the reaction unit.

7. The method of claim 6, wherein the nitrogen source supply step comprises continuously supplying the nitrogen source at a concentration of 1 to 10 g/L per 1 g/L of the aerobic methanotroph.

8. The method of claim 6, wherein the nitrogen source is at least one selected from the group consisting of potassium nitrate (KNO₃), sodium nitrate (NaNO₃), ammonium chloride (NH₄Cl), ammonium sulfate ((NH₄)₂SO₄), yeast extract, urea, peptone, tryptone, or beef extract.

9. The method of claim 1, wherein the product comprises at least one selected from the group consisting of ectoine, methanol, PHA (polyhydroxyalkanoate), PHB (polyhydroxyvalerate), PHBV (Poly(3-hydroxybutyrate-co-3-hydroxyvalerate)), microbial protein, biodiesel precursor, lactic acid, butyric acid, acetic acid, muconic acid, succinic acid, 3-hydroxypropionic acid, 2,3-butanediol, putrescine, cadaverine, or sesquiterpene.

10. The method of claim 1, further comprising a recycling step of re-supplying the medium separated from the product back into the reaction unit.

11. The method of claim 1, wherein the cultivation step is carried out until all the methane contained in the reaction unit is consumed.

12. The method of claim 1, wherein the final gas consists only of water and carbon dioxide.


SEQUENCE LISTING (12 sequences)

SEQ ID NO: 1 - DNA, 1274 bp, Methylomicrobium alcaliphilum
  source 1..1274: mol_type = genomic DNA; strain = 20Z
atgaaagtctttgaacaatgggaatccgaaattcgcggctactgccgtgtgtatcctaccgttttcaaatcggcctccaatgcccgtcaagtggatgagtcgggaaaatcctatatcgacttcttcgcaggcgccggtgtactcaatttcggccacaacaacccgttgatgaaaaaagccttgatcgattttatcgaggcggatggcgtagcccacagtctggacacctacaccacggcgaaacgcgattttatcgaagccttcgccaattccgtgcttaaaccgcgaaaaatgaattacaaaatgcaattcatgggaccgaccggaactaatgcggtcgaaaccgcgctgaaattggcgcgcaaggtcaccgggcgccgttcgattattgcgttcaatcacggttttcacggcatgacactgggctcgctggcctgcacggcaaaccaatatttccgcaatgccgccggcgtacccctggaatatgttcgtcatgaacccttcggctgcgaaaagccttgtatcggttgccaattaggctgtggcctggaaactatcgatcagttgcgcgcgcaattttccgattcgtcgagcggcctggaaccgccggcggcgtttttagtcgaaccgattcaagccgaaggcggcgtcaatgtagccagtcgggaatggttgcacgccgtgcaaaaaatggcgcacgatctaggcgcgctattgatttttgacgacatccaggcaggttgcgggcgcaccggtcaatatttcagttttgacggcatggatttggaaccggacattatcacattagccaaaggcatcggcggcttcggcacgccgctggcgatgaatttggtcaagcctgaacacgaccagcattggcagcccggcgaacataccggcactttcagaggtcaaggcttctccttcgtcgcgggcaaagtggcgctgtcctatttcgacgatgacgcattgatgaacgatgtcaagctgaaaggcactaagatgcagcagcacctcgaagacttggcggaacgcttcggtcaagggcggtttcaagtgcgcggcaaaggcatgatgcaagggctggacatcgccgatggcgccttggcgaaaaacatcgtcaatctttgcttcgaacgcggactactactcagcgcctgcggaacgggcggcaaggtcatcaaaatgattccgccgctaacgattccggaagccgatctgctcgaaggtttaacgattttctcgggcgtcttagcggatgctttggaggcctt

SEQ ID NO: 2 - DNA, 1588 bp, Methylomicrobium alcaliphilum
  source 1..1588: mol_type = genomic DNA; strain = 20Z
gcaaggagggttactcaatggctaccacgctccagaaactgtgaaagtattcaagattatgaattaaccatgaagaacatgaagaattagattgttgactttataggctttttcttcatggtgaataaattttttatgatgtattcactaacttacggcctcaacagcgtgggagcgatgaataatgatattcctaatttagcttattcaacggagaacgaaactaatgagcatacaagttattaatccatccaacggcgaatcattgggcataagcccggaactcgacgccgccgctcgcgatgctgcgctcgatcgcgcgcaaatagcctatgccgattggagaaaccaatcctttgcgcaacgcgcgcaaatgttgcgtaaaatcgctcagcgtttacgcgatgatgtagaatcgctggcaccgctgatgaccttggagatgggcaaaccgatcaaggaggcccgtgctgaagtgatgaaggcagcgctctgcgccgaccattacgccgaacatggcgagagctatcttgccccggatacattggcatctgacgccagtcttagctatgtgcaatatctgcctttgggcgttgtactgggcattttgccttggaacgcaccattttggctggcttttcgcttttgcgccccggcattgatggccggaaacacctgcctgatgaagcatgacccgcatgttccggcctgcgccgaagcaatcgcgcggctatttgagcagagcggagccccggccggtctattgcaaaatctacccttgtcgaacaccgatgtagaagcggtcattcgcgatccacgcgtacaagccgtctcgttgaccggttcggatagagccggcactgtcgtggcctcgatcgccggtgctgaaatcaaaccggtcgttctggaattgggcggctccgatccctgcatcgtactggccgatgcagatctggacaaagccgccgatgtgattacgctgtcgcgcatcatcaatgccggccaatcttgtatcgccgccaaacgcatcattgtcgaagccggcattcatgatgaactcgtgacacggcttgaagaacgtctaagcaagttgaaactgggcgacccgaatgaggaaagcacagacatcggccccttggcgcgagaagacttgcggctaaacctgcatcgtcaggttcaagaaacgattgccgccggcgcacaatgccgactcggcggaaaaataccggacgggccgggctatttttatcccgtgaccttgctaaccggcgttacgaacgacatgacggccgcccgagaagaaaccttcggcccggtcgctgtagttattaaagccgaagatcaagatgaagcgatgcgcatagccaacgacagtcaatatggtctagccgccagcatctggacggaacggtcgcgaggcgaagcgctggctcggcaattggaaaccggccaagttgcggtcaacggcatcgtcaaaaccgaccccagactaccaagcggcggcgtcaagcgctcaggactcggccgcgaactggggccgcacggcatgcacgaatttgtcaacgcgcagcaggtttggctgggttaa

SEQ ID NO: 3 - DNA, 45 bp, synthetic construct
  source 1..45: mol_type = other DNA
tgacatgattacgccaagcttatgaaagtctttgaacaatgggaa

SEQ ID NO: 4 - DNA, 29 bp, synthetic construct
  source 1..29: mol_type = other DNA
ccctccttgcaaggcctccaaagcatccg

SEQ ID NO: 5 - DNA, 32 bp, synthetic construct
  source 1..32: mol_type = other DNA
tggaggccttgcaaggagggttactcaatggc

SEQ ID NO: 6 - DNA, 43 bp, synthetic construct
  source 1..43: mol_type = other DNA
aaaacgacggccagtgaattcttaacccagccaaacctgctgc

SEQ ID NO: 7 - DNA, 20 bp, synthetic construct
  source 1..20: mol_type = other DNA
atgcttccggctcgtatgtt

SEQ ID NO: 8 - DNA, 20 bp, synthetic construct
  source 1..20: mol_type = other DNA
tccggatcgctgattacgac

SEQ ID NO: 9 - DNA, 20 bp, synthetic construct
  source 1..20: mol_type = other DNA
gcgtcgaaggcgataaaacc

SEQ ID NO: 10 - DNA, 20 bp, synthetic construct
  source 1..20: mol_type = other DNA
taagcccactgcaagctacc

SEQ ID NO: 11 - DNA, 20 bp, synthetic construct
  source 1..20: mol_type = other DNA
gcttcgaacgcggactacta

SEQ ID NO: 12 - DNA, 20 bp, synthetic construct
  source 1..20: mol_type = other DNA
cgctgttgaggccgtaagtt